(19) United States
(12) Patent Application Publication
(10) Pub. No.: US 2024/0228454 A1
(43) Pub. Date: Jul. 11, 2024

(54) RING CLOSURE OF BENZOQUINONES CONTAINING AN UNSATURATED SIDE CHAIN USING A BASE IN TWO-PHASE SYSTEM

(71) Applicant: DSM IP ASSETS B.V., HEERLEN (NL)

(72) Inventors: Werner BONRATH, Kaiseraugst (CH); Rolf KUENZI, Kaiseraugst (CH)

(21) Appl. No.: 18/557,648

(22) PCT Filed: Apr. 26, 2022
(86) PCT No.: PCT/EP2022/061094

§ 371 Date: Oct. 27, 2023

(30) Foreign Application Priority Data
Apr. 28, 2021 (EP) 21170911.8

(51) Int. Cl. C07D 311/72; C07D 311/92

(52) U.S. Cl. CPC C07D 311/72; C07D 311/92

(57) ABSTRACT

The present invention relates to the formation of compound of the formula (I) by a ring closure reaction of the compound of the formula (II) in the presence of a base in a two-phase system. It has been found that this reaction is very efficient and offers for example an efficient pathway for the synthesis of 3,4-dehydro-α-tocotrienol respectively a-tocotrienol and a-tocopherol.

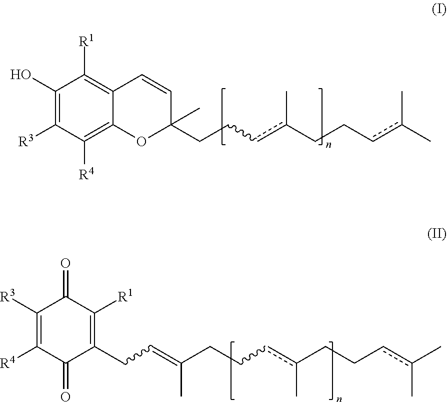

### TECHNICAL FIELD

The present invention relates to the field of the synthesis of chromanes and chromenes, particularly, of 3,4-dehydrotocopherols, 3,4-dehydrotocotrienols, tocopherols and tocotrienols.

### BACKGROUND OF THE INVENTION

An important class of chromane compounds are vitamin E and its esters. A synthetic pathway for chromanes is via their corresponding chromenes.

There exist different routes for the formation of chromenes.

Schudel, Mayer, Isler, Helv. Chim. Acta 46, 2517-2526 (1963) discloses the formation of 3,4-dehydro tocotrienol by a ring closure reaction of geranyl-geranyl trimethyl benzoquinone in the formation of chromenes by ring closure in pyridine as reaction medium. The reaction mixture obtained is a complex mixture and the isolation of the desired product requires a complex derivatisation procedure forming the dehydro tocotrienol-p-phenylazobenzoate for separation and purification by crystallisation. For this procedure the very expensive and highly toxic chemical 4-(phenylazo)benzoyl chloride is used and, therefore, is very disadvantageous.

WO 2015/028643 A1 discloses the formation of chromenes by Au(I) or Ag(I) catalysed intramolecular hydroarylation of chiral aryl alkynes. Gold and silver catalyst are very expensive.

Kabbe and Heitzer, Synthesis 1978, 12, 888-889 discloses that using the known synthetic pathway for vitamin E (i.e. α-tocopherol) from TMHQ and isophytol is not suitable for the synthesis of tocotrienols (i.e. from TMHQ and geranyllinalool) because the isoprenoid side chain undergoes acid-catalyzed secondary ring closure reactions.

### SUMMARY OF THE INVENTION

Therefore, the problem to be solved by the present invention is to offer a process to provide chromenes and chromanes in a manner which is much simpler that a process which would involve a complex derivatisation, purification and chemical transformation to the yield finally the desired product.

This problem has been solved by the process according to claim 1. It has been surprisingly found that a two-phase systems in presence of a base is very suitable for this purpose and allows an easy isolation of the desired compound of the formula (I). It has been particularly found that catalyst which are strongly basic are particularly suitable as base for said ring closure reaction.

This process offers a very favourable synthetic pathways to chromanes of the formula (III) or (IV) according to the claim 11 or 12.

Further aspects of the invention are subject of further independent claims. Particularly preferred embodiments are subject of dependent claims.

### DETAILED DESCRIPTION OF THE INVENTION

In a first aspect, the present invention relates to a process of manufacturing the compound of the formula (I)

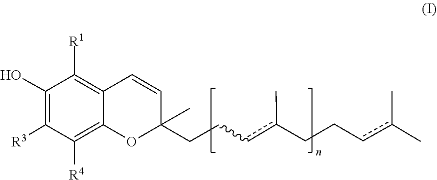

comprising a ring closing step of the compound of the formula (II)

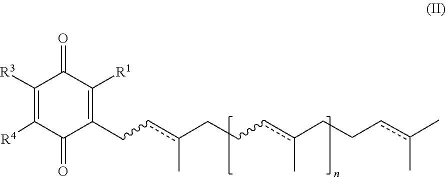

in the presence of a base in a two-phase system to yield the compound of the formula (I),
wherein
   n=0 or 1 or 2 or 3 or 4 or 5 or 6 or 7 or 8 or 9 or 10 or 11 or 12;
   R¹ represents hydrogen or methyl groups;
   R³ and R⁴
      either represent independently from each other hydrogen or methyl group or methoxy group
      or represent together a —CH—CH—CH— and form an aromatic group; any bond having dotted line ( [symbol] ) represents independently from each other either a carbon-carbon single bond or a carbon-carbon double bond; and
   any wavy line represents independently from each other a carbon-carbon bond and which when linked to the carbon-carbon double bond is either in the Z or in the E-configuration.

For sake of clarity, some terms used in the present document are defined as follows:

In the present document, a “C_{x-y}-alkyl” group is an alkyl group comprising x to y carbon atoms, i.e., for example, a C₁₋₃-alkyl group is an alkyl group comprising 1 to 3 carbon atoms. The alkyl group can be linear or branched. For example —CH(CH₃)—CH₂—CH₃ is considered as a C₄-alkyl group.

In case identical labels for symbols or groups are present in several formulae, in the present document, the definition of said group or symbol made in the context of one specific formula applies also to other formulae which comprises the same said label.

The term “independently from each other” in this document means, in the context of substituents, moieties, or groups, that identically designated substituents, moieties, or groups can occur simultaneously with a different meaning in the same molecule.

In the present document, any dotted line in formulae represents the bond by which a substituent is bound to the rest of a molecule.

In the present document any bond having dotted line ( [symbol] ) in a chemical formula represents independently from each other either a carbon-carbon single bond or a carbon-carbon double bond.

Any wavy line in any formula of this document represents a carbon-carbon bond and which when linked to the carbon-carbon double bond is either in the Z or in the E-configuration. It is preferred in all molecules that the carbon-carbon double bond is in the E-configuration.

The “pKₐ” is commonly known as negative decadic logarithm of the acid dissociation constant (pKₐ=−log₁₀ Kₐ). When the organic acid has several protons the pKₐ as used in this document relates to the dissociation constant of the last proton. For example, for a base having two basic sites the “pkₐ” relates to pKₐ2. The pKₐ are measured at standard temperature and pressure.

### Compound of the Formula (II)

The compounds of the formula (II) are substances as well as their synthesis is known to the person skilled in the art.

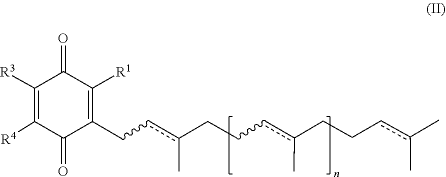

In one preferred embodiment, the substituent R³ and R⁴ represent methoxy groups. In this embodiment any bond having dotted line ( [symbol] ) represents preferably a carbon-carbon double bond, which is preferably in the E-configuration.

Ubiquinones are important representatives of this embodiment. The ubiquinones are denoted according to the number of isoprenoid groups in the side chain as ubiquinone-2 (n=0), ubiquinone-3 (n=1), ubiquinone-4 (n=2), ubiquinone-5 (n=3), ubiquinone-6 (n=4, ubiquinone-7 (n=5), ubiquinone-8 (n=6), ubiquinone-9 (n=7) and ubiquinone-10 (n=8). The ubiquinones are also known under the old term coenzyme Q. Ubiquinone-10 (n=8) (=coencyme Q10) is a particular preferred species of this embodiment.

In another preferred embodiment, the substituent R³ and R⁴ represent either H or methyl group. It is preferred that R³=R⁴=CH₃

It is particularly preferred that R¹=R³=R⁴=CH₃.

It is preferred that n=2. It is further preferred that all bonds having dotted line ( [symbol] ) in formula (II) are carbon-carbon double bonds, and preferably all in the E-configuration.

It is preferred in this embodiment that the compound of the formula (II) is the compound of the formula (II-BB)

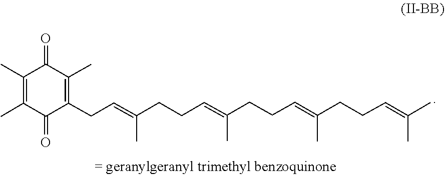 [SMILES: CC(C)=CCC/C(C)=C/CC/C(C)=C/CC/C(C)=C/CC1=C(C)C(=O)C(C)=C(C)C1=O]

In another preferred embodiment, the substituent R³ and R⁴ represent together a —CH—CH—CH— and form an aromatic group. The compounds of this embodiment are represented by

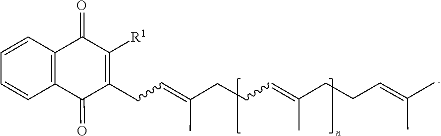

In this embodiment, R¹ represents preferably a methyl group.

Vitamin K1 (phylloquinone) is one example of this embodiment.

Menaquinones (MK), also known as vitamin K2, are further important representatives of this embodiment.

Any bond having dotted line ( [symbol] ) represents preferably a carbon-carbon double bond, which is preferably in the E-configuration

The menaquinones are denoted according to the number of isoprenoid groups in the side chain as MK-2 (n=0), MK-3 (n=1), MK-4 (n=2), MK-5 (n=3), MK-6 (n=4), MK-7 (n=5), MK-8 (n=6), MK-9 (n=7), MK-10 (n=8), MK-11 (n=9), MK-12 (n=10) and MK-13 (n=11).

MK-4 (n=2) is a particular preferred species of this embodiment.

In case that any of the bond having dotted line ( [symbol] ) represents a carbon-carbon double bond, the formation of secondary ring formation a risk of would be expected by the person skilled in the art. As this has not been observed, it is particularly preferred that at least one of the bonds having dotted lines represents a carbon-carbon double bond. Therefore, this process particularly leads to α-tocotrienol, which has three double bonds in the side chain. αTocotrienol is an important compound in natural vitamin E.

### Base

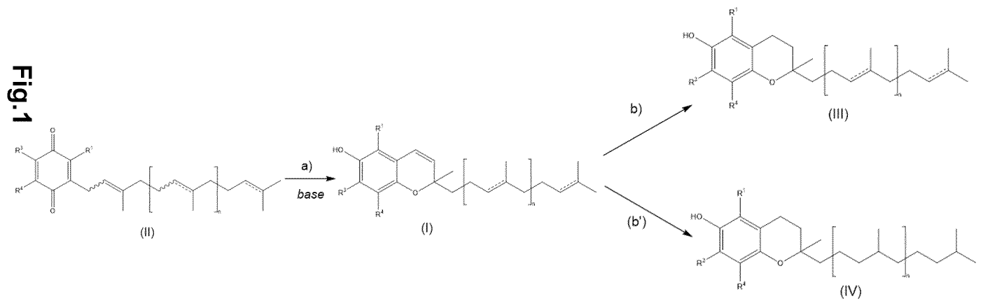

Said process comprises a ring closing step of the compound of the formula (I) in the presence of a base (“base”) to yield the compound of the formula (I) as depicted as step a) in the reaction scheme of FIG. 1.

The base is preferably a hydroxide or a carbonate, preferably a hydroxide, of an alkali metal or an earth alkali metal, particularly an alkali metal hydroxide.

Furthermore preferred is that the base is an organic amine, particularly an organic tertiary amine.

Not all bases work equally well for the present invention. It is preferred that said base is not pyridine. It has been shown that the conjugated acids of said base having a pKₐ of between 8.6 and 15.7, particularly of between 9 and 15.7, measured in water, are particularly suitable. This means that said base has preferably a pK_{b} of between 5.4 and 0, particularly of between 5 and 0.

A few examples of pkₐ of the corresponding acids for:

| Base | pka¹ |
|---|---|
| 1,5-diazabicyclo[4.3.0]non-5-ene (=DBN) | 13.42³ |
| 1,8-diazabicyclo[5.4.0]undec-7-ene (=DBU) | 12², 13.28³ |
| 1-azabicyclo[2.2.2]octane (=quinuclidine) | 11², 10.95³ |
| 4-(dimethylamino)pyridine (=DMAP) | 9.2², 9.7³ |
| sparteine | 9.45² |
| 1,4-diazabicyclo[2.2.2]octane (=DABCO) | 8.8², 8.82³ |
| NaOH | 15.7² |
| Na₂CO₃ | 10.4³ |
| morpholine⁴ | 8.36², 8.49³ |
| triethanolamine⁴ | 7.76³ |
| pyridine⁴ | 5.23², 5.23³ |

¹pka of the corresponding conjugated acid
²H. Ripin; D. A. Evans (2002). “pKₐ's of Nitrogen Acids” https://organicchemistrydata.org/hansreich/resources/pka/pka_data/evans_pKa_table.pdf
³https://www.aatbio.com/data-sets/pka-and-pkb-reference-table
⁴base which are less preferred (pkₐ < 8.6)

In one embodiment said base is an organic amine, particularly selected from the group consisting of 4-dimethylaminopyridine (=DMAP), 1,8-diazabicyclo[5.4.0]undec-7-ene (=DBU), 1,5-diazabicyclo[4.3.0]non-5-ene (=DBN), 1,4-diazabicyclo[2.2.2]octane (=DABCO), 1-azabicyclo[2.2.2]octane (=quinuclidine) and sparteine, preferably from the group consisting of 4-dimethylaminopyridine (=DMAP), 1,8-diazabicyclo[5.4.0]undec-7-ene (=DBU) and 1-azabicyclo[2.2.2]-octane (=quinuclidine).

The base is particularly not a hydride, such as sodium hydride, as hydrides form molecular hydrogens when contacted with the compounds of the formula (II). The formation of hydrogen leads to significant safety risks in the ring closing step and generally in processing.

In another embodiment the base is preferably a hydroxide or a carbonate, preferably a hydroxide, of an alkali metal or an earth alkali metal, particularly an alkali metal hydroxide. In this embodiment, most preferably, the base is NaOH or KOH.

In a still another embodiment, the base is potassium fluoride on alumina (KF/Al₂O₃).

### Two-Phase System

The ring closing step of the compound of the formula (II) is performed in the presence of a base in a two-phase system.

Said two-phase system is a system for the reaction with two different phases. The two-phase system particularly consists of a liquid phase and a solid or another liquid phase.

It is preferred that one of the two phases comprises a hydrocarbon, preferably toluene.

The compound of the formula (II) is particularly dissolved in said hydrocarbon, preferably in toluene.

The base is preferably in a different phase than the compound of the formula (II).

In one of the embodiment said two-phase system consists of two liquid phases, preferably an aqueous phase and an organic phase which is not miscible with the aqueous phase. Said organic phase comprises particularly a hydrocarbon, preferably toluene.

Said aqueous phase preferably comprises the base.

In another embodiment said two-phase system consists of a liquid phase and a solid phase.

It is preferred that the base is in form of a solid. Hence, the solid phase particularly is or comprises the base. The base can be supported by a solid carrier. The base can be absorbed or adsorbed, or chemically bound to the solid carrier such as for example to alumina, silica, carbon, carbonates or silicates or other minerals.

The liquid phase in this embodiment is either the compound of the formula (II) or a liquid organic phase which comprises an organic solvent. The organic solvent is particularly a hydrocarbon, preferably toluene.

Hence, as a solid phase, a solid hydroxide or a carbonate, preferably a hydroxide, of an alkali metal or an earth alkali metal, particularly an alkali metal hydroxide can be used. Said solid hydroxide or carbonate is preferably used as solid having high surface area, which is obtained particularly by a mechanical process such as by grinding, milling or pulverization or by physicochemical process such as suitable precipitations or crystallizations. It has been found that the higher the surface area (defined for example by m²/g) of the solid base is the better said solid base is suited for the purpose of the invention.

In a particular preferred embodiment said solid base is potassium fluoride on alumina (KF/Al₂O₃). Potassium fluoride on alumina is a well-known base which is of broad use for organic syntheses, as disclosed by B.E. Blass in Tetrahedron 58 (2002), 9301-9320.

20) When the base is used as solid, it is preferred that a phase transfer agent is used, particularly a quaternary ammonium salt, particularly of the formula [NR₄]X wherein R is a C₂₋₁₈-alkyl group, particularly a C₃₋₈-alkyl group, and X is a halide. Preferably, the phase transfer agent is a tetrabutyl ammonium halide, particularly tetrabutyl ammonium bromide. Said phase transfer agent is preferably used in amounts of 0.1 to 10 mol %, particularly of 0.5 to 2 mol %, relative to the compound of formula (II).

It has been observed that particularly the two-phase systems which consists of a liquid phase and a solid show very high yields at high conversions. Therefore, a two-phase system consisting of a liquid phase and a solid is preferred.

The use of a two-phase system for the ring closing reaction is very advantageous as the phases can be easily separated from each other, which results in better separation not only of the starting materials but also the reaction product. Hence, the use of such two-phase system for the reaction at issue is advantageous for the working up respectively isolation of the reaction product. Furthermore, it simplifies the recycling of starting materials and is advantageous in continuous reaction modes which is the preferred way of producing the compound of the formula (I) in industrial scale. When the base is provided as solid phase, it can be easily provided in a form of a larger object typically in form of a structured packing element which might be a part of the reactor in which the reduction takes place or an element which is inserted into said reactor. This structured packing element may be a dumped packing, a knit, an open-celled foam structure, preferably made of plastic, for example polyurethane or melamine resin, or ceramic, or a structured packing element, as already known in principle, i.e. by its geometric shape, from distillation and extraction technology. It is also possible that the solid base is comprised in a containment having porous walls such as for example a net or mesh which has suitable hole or mesh diameters in allowing the reaction medium to be transmitted, however, in avoiding the solid base of doing so. Useful structured packing elements are in particular metal fabric packings and wire fabric packings, for example of the design Montz A3, Sulzer BX, DX and EX. Instead of metal fabric packings, it is also possible to use structured packings made of other woven, knitted or felted materials. Further useful structured packings are of flat or corrugated sheets, preferably without perforation, or other relatively large orifices, for example corresponding to the designs Montz BI or Sulzer Mellapak. The structured packings made of expanded metal are also advantageous, for example structured packings of the type Montz BSH. In such a case, the base can be easily removed from the reactor and replaced by fresh base, particularly when the reaction is run in a reactor used in a continuous reaction mode.

It has been shown that the base can be used for the ring closing step particularly in amounts of a molar ratio of base/compound of the formula (II) of more than 1, particular between 2 and 1.1.

However, it has been surprisingly found, that the base can be used also in catalytical amounts, i.e. that the base is not present in stoichiometric amounts relative to the compound of the formula (II), but in significantly lower amounts, i.e. the molar ratio of the basic catalyst to the compound of the formula (I) is 1:1′000 to 1:5, particularly 1:1′000 to 1:8, more particularly 1:100 to 1:10.

The ring closing step is typically performed under stirring preferably at a temperature of between 40 and 200° ° C., preferably between 90 and 150° C., more preferably at the reflux temperature of the organic solvent when an organic solvent is used, and/or at a pressure of between 1 bara and 10 bara. It is further preferred that this reaction is performed under inert atmosphere, preferably under nitrogen.

It has been shown that said process smoothly yields the compound of the formula (I).

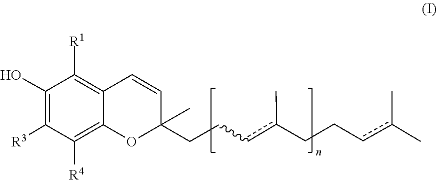

Particularly, said process allows the isolation of the desired compound of the formula (I) in a simple way, i.e. without the need of any complex derivatization followed by purification by crystallization and chemically transforming the derivate finally to the desired compound as it is the case in the process as disclosed by Schudel, Mayer, Isler, Helv. Chim. Acta 46, 2517-2526 (1963).

Particularly preferred embodiments of the compound of the formula (I) are the compounds of the formula (I-A), (I-B) and (I-C), preferably the compound of the formula (I-AA), (I-BB), (I-CC1) and (I-CC2):

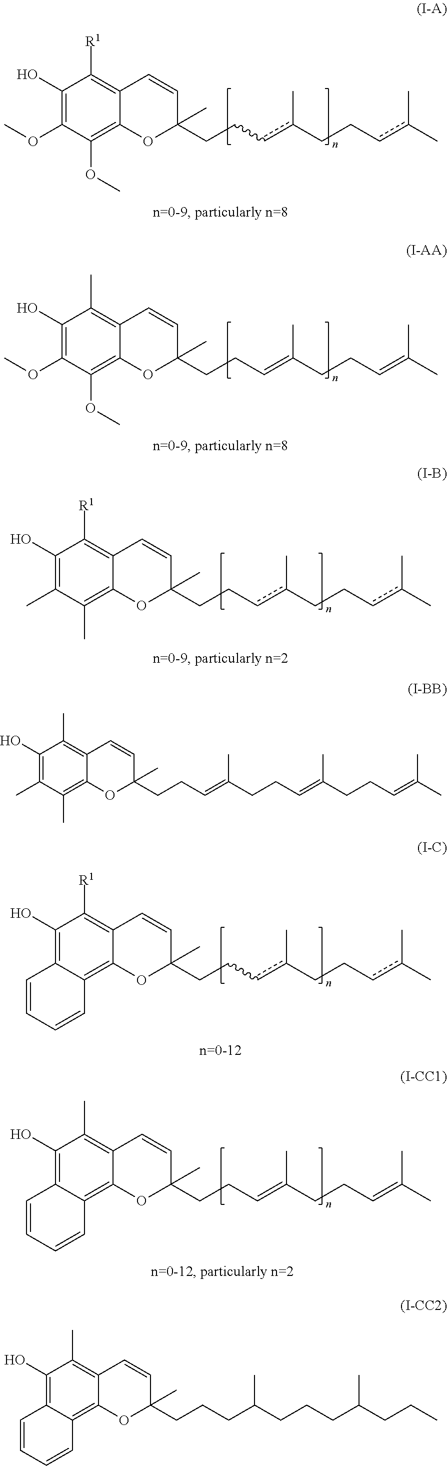

Very preferred compounds are the compounds of formula (I-As)

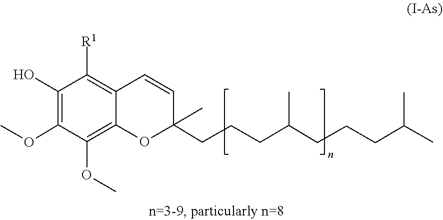

Very preferred compounds are the compounds of formula (I-Cis)

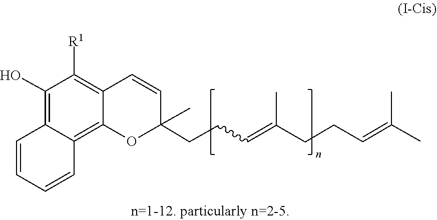

The compound of the formula (I) obtained as shown above can be hydrogenated by means of a hydrogenation agent.

In one embodiment, in this hydrogenation only the carbon-carbon double bond in the ring is hydrogenated whereas the olefinic carbon-carbon double bonds are not hydrogenated (“partial hydrogenation”), so that the hydrogenation leads to the compound of the formula (III) has been depicted in FIG. 1.

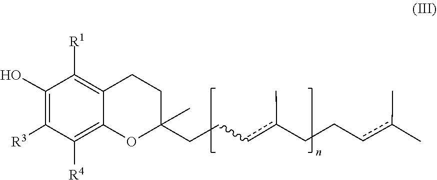

Particularly preferred embodiments of the compound of the formula (III) are the compounds of the formula (III-A), (III-B) and (III-C), preferably the compound of the formula (III-AA), (III-BB), (III-CC1) and (III-CC2):

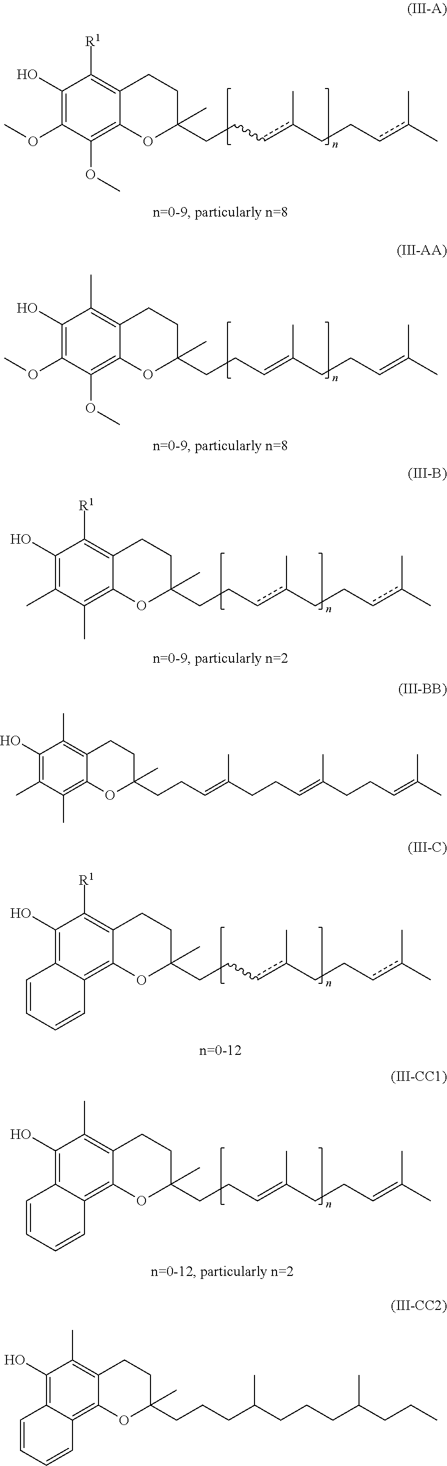

Very preferred compounds are the compounds of formula (III-Cis)

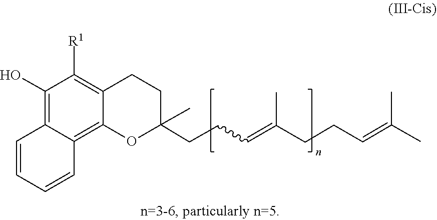

In another embodiment, in this hydrogenation all olefinic carbon-carbon double bonds are hydrogenated (“complete hydrogenation”) so that the hydrogenation leads to the compound of the formula (IV) has been depicted in FIG. 1.

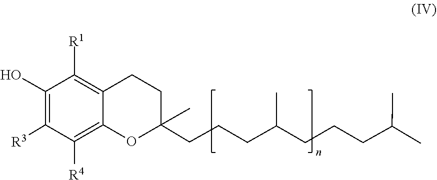

Particularly preferred embodiments of the compound of the formula (IV) are the compounds of the formula (IV-A), (IV-B) and (IV-C), preferably the compound of the formula (IV-A), (IV-BB) and (IV-CC):

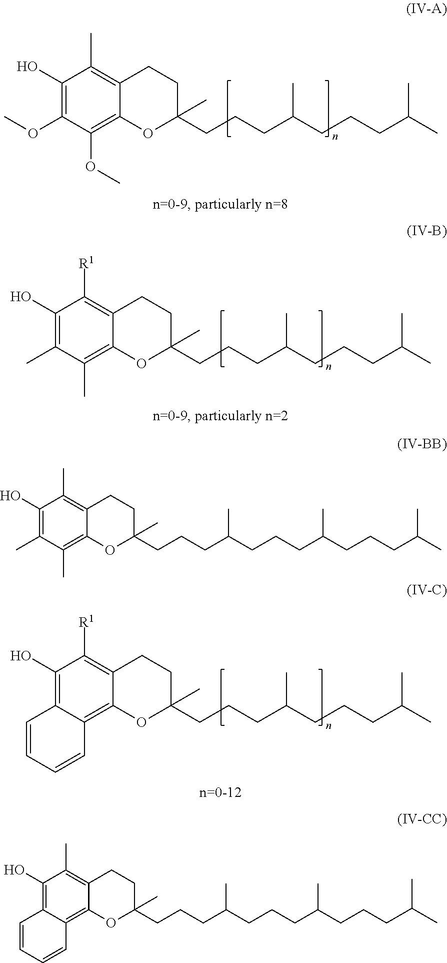

Very preferred compounds are the compounds of formula (IV-Cs)

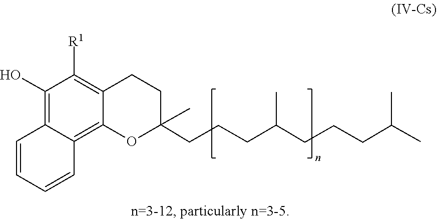

Hence, in a further aspect, the present invention also relates to a process The process of manufacturing a compound of the formula (III)

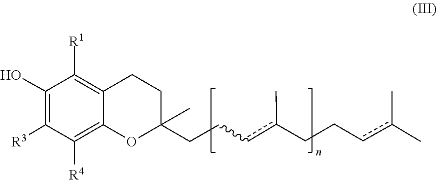

comprising the steps
a) manufacturing the compound of the formula (I) by a process as discussed above in great detail;

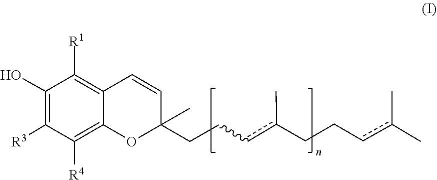

wherein any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration;
b) partially hydrogenating the compound of the formula (I) by means of a hydrogenating agent suitable for partial hydrogenation to yield the compound of the formula (III).

The hydrogenating agent used in step b) is a hydrogenating agent which only hydrogenates the carbon-carbon double bond of the ring in formula (I). Particularly suitable as hydrogenating agent is sodium/ethanol such as described by Schudel, Mayer, Isler, Helv. Chim. Acta 46, 2517-2526 (1963), particularly in the last paragraph on page 2524.

Hence, in a further aspect, the present invention also relates to a process of manufacturing a compound of the formula (IV)

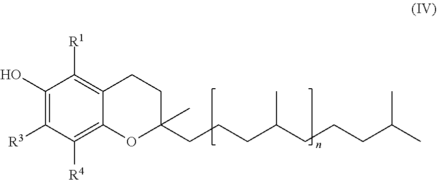

comprising the steps
a) manufacturing the compound of the formula (I) by a process as discussed above in great detail

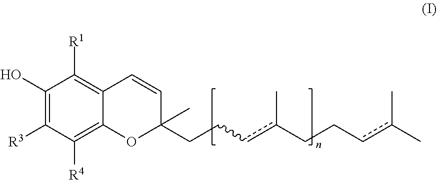

wherein any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration;
b′) hydrogenating the compound of the formula (I) by means of a hydrogenating agent to yield the compound of the formula (IV).

The hydrogenating agent used in step b′) is a hydrogenating agent which hydrogenates all olefinic carbon-carbon double bond of the ring in formula (I). Particularly suitable as hydrogenating agent is hydrogen in the presence of a transition metal from the groups 7, 8, 9 or 10, particularly selected form the group consisting of Pd, Pt, Rh, Ru, Mn, Fe, Co, and Ni, more preferably Pd.

The heterogenous transition metal catalyst is preferably a heterogenous supported transition metal catalyst.

In this embodiment, the transition metal is supported on a carrier, i.e. palladium is attached to/or deposited on a carrier. The carrier is a solid material.

Preferably said carrier is carbon or an inorganic carrier. Preferred inorganic carriers are oxides or carbonates. Preferred oxides are oxides of Si, Al, Ce, Ti or Zr, particularly of Al or Si. Particularly preferred are silicon dioxide, alumina and titanium dioxide and ceria.

In case the support is Ce, the preferred oxide is CeO₂. Preferably, the oxide of Al is Al₂O₃ and AlO(OH). Particularly preferred is Al₂O₃.

It is preferred to perform the hydrogenation under pressure, particularly under a hydrogen pressure of 2 to 20 bar. It is further preferred to perform the hydrogenation at a temperature between 0° ° C. and 100° C.

The two-phasic composition comprising the compound of the formula (II) and the base itself is also an object of the present invention.

Hence, in a further aspect, the invention relates to a two-phasic composition comprising
i) a compound of the formula (II)

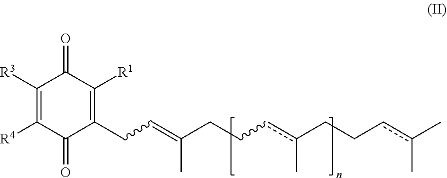

wherein
   n=0 or 1 or 2 or 3 or 4 or 5 or 6 or 7 or 8 or 9 or 10 or 11 or 12;
   R¹ represents hydrogen or methyl groups;
   R³ and R⁴
      either represent independently from each other hydrogen or methyl group or methoxy group
      or represent together a —CH—CH—CH— and form an aromatic group;
   any bond having dotted line ( [symbol] ) represents independently from each other either a carbon-carbon single bond or a carbon-carbon double bond; and
   any wavy line represents independently from each other a carbon-carbon bond and which when linked to the carbon-carbon double bond is either in the Z or in the E-configuration;
and
ii) a base in another phase than the compound of the formula (II).

The compound of the formula (II) and the base as well their preferred embodiments have been already discussed above in great detail for the process.

Within this invention, it has been found that the reaction system based on two phases can be used for an efficient ring closure in the above ring closure step.

Hence, in a further aspect, the invention relates to a use of a base in a different phase than the compound of the formula (II) for the ring closure reaction of the compound of the formula (II) to yield the compound of the formula (I)

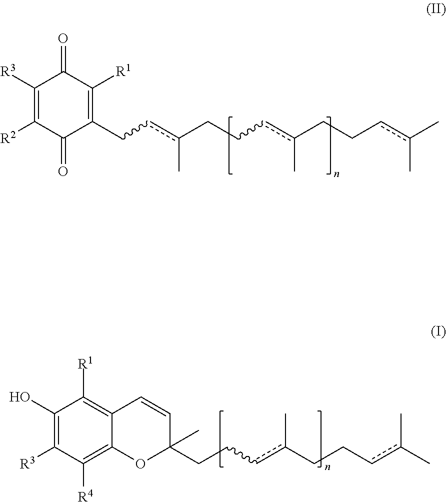

wherein
   n=0 or 1 or 2 or 3 or 4 or 5 or 6 or 7 or 8 or 9 or 10 or 11 or 12;
   R¹ represents hydrogen or methyl groups;
   R³ and R⁴
      either represent independently from each other hydrogen or methyl group or methoxy group
      or represent together a —CH—CH—CH— and form an aromatic group;
   any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration.

The compound of the formula (II), the base, the two phases and ring closure step as well their preferred embodiments have been already discussed above in great detail for the process.

It has been further found that the compound of the formula (I-A) or (I-C) or (III-A) or (III-C) or (IV-A) or (IV-C) has antioxidative properties.

Hence, in a further aspect, the invention relates to the use of the compound of the formula (I-A) or (I-C) or (III-A) or (III-C) or (IV-A) or (IV-C) as an antioxidant.

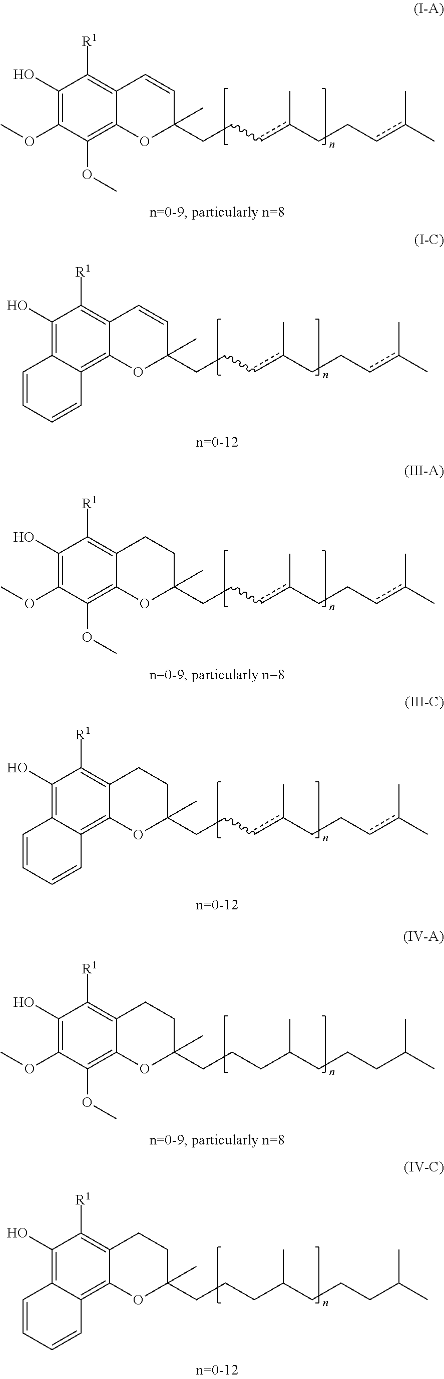

wherein R¹ represents hydrogen or methyl groups.

The compounds of the formula (I-A) or (I-C) or (III-A) or (III-C) or (IV-A) or (IV-C) as well their preferred embodiments have been already discussed above in great detail for the process.

Several compounds disclosed in this document are new. Due to their suitability for the disclosed processes and uses said compound are not only new but also inventive.

Hence, in a further aspect, the invention relates particularly to the compounds of the the formula (I-As) or (I-Cis) or (III-Cis) or (IV-Cs)

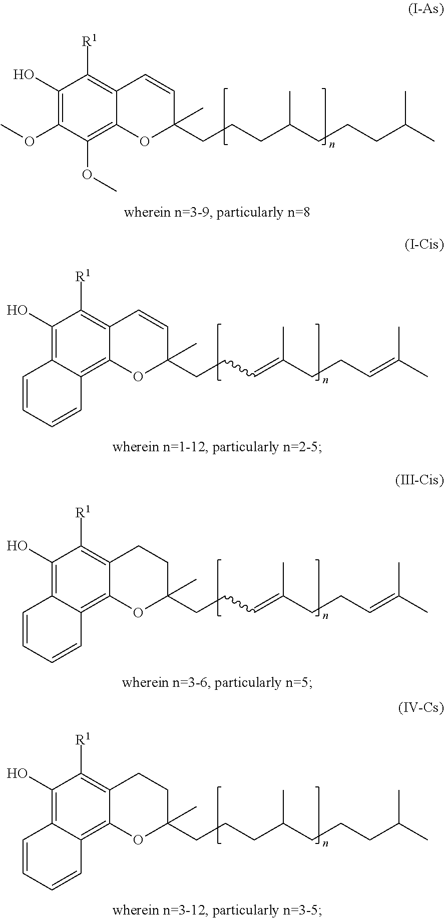

and wherein
R¹ represents hydrogen or methyl groups;
any bond having dotted line ( [symbol] ) represents independently from each other either a carbon-carbon single bond or a carbon-carbon double bond; and any wavy line represents independently from each other a carbon-carbon bond and which when linked to the carbon-carbon double bond is either in the Z or in the E-configuration.

### EXAMPLES

The present invention is further illustrated by the following experiments.

### Experimental Series 1: Two-Phasic System Liquid/Solid: Toluene/Solid NaOH

In a first series, 0.46 g (1.098 mmol) geranylgeranyl trimethyl benzoquinone (purity 97%) and 6 ml toluene and 4.7 mg ground solid NaOH (0.1098 mmol, 10 mol % (relative to geranylgeranyl trimethyl benzoquinone)) in the presence of 3.5 mg of tetrabutylammonium bromide (1 mol % (relative to geranyl-geranyl trimethyl benzoquinone)) were added and stirred under reflux (110° C.) for the reaction time given in table 1 to yield the 2,5,7,8-tetramethyl-2-(4,8,12-tri-methyltrideca-3,7,11-trien-1-yl)-2H-chromen-6-ol (3,4-dehydro-α-tocotrienol) in conversion and yield as indicated in table 1.

TABLE 1
Solid NaOH use as solid phase base in a two-phase system solid/liquid.

| | Base/quinone | Reaction | Conversion | Yield |
|---|---|---|---|---|
| Example | [mol/mol] | Time [h] | [%] | [%] |
| 1 | 1/10 | 1 | 86 | 82.4 |
| 2 | 1/10 | 16 | 95 | 92.6 |
| 3 | 1/10 | 36 | 96 | 93.3 |

### Experimental Series 2: Two-Phasic System Liquid/Solid: Toluene/Potassium Fluoride on Alumina (KF/A|203)

In a second series, 0.46 g (1.098 mmol) geranylgeranyl trimethyl benzoquinone (purity 97%) and 6 ml toluene and 46 mg of the base potassium fluoride on alumina (KF: 40% by weight) and tetrabutylammonium bromide (1 mol % (relative to geranylgeranyl trimethyl benzoquinone)) were added and stirred under reflux (110° C.) for 48 hours to yield the 2,5,7,8-tetramethyl-2-(4,8,12-trimethyl-trideca-3,7,11-trien-1-yl)-2H-chromen-6-ol (3,4-dehydro-α-tocotrienol) in conversion and yield as indicated in table 2.

TABLE 2
Potassium fluoride on alumina (KF/Al₂O₃) as solid phase
base in a two-phase system solid/liquid

| Example | Base/quinone [g/g] | Conversion [%] | Yield [%] |
|---|---|---|---|
| 4 | 1/10 | 95 | 93 |

### Experimental Series 3: Two-Phasic System Liquid/Liquid: Toluene/Aqueous Base Solution

In a third series, 0.46 g (1.098 mmol) geranylgeranyl trimethyl benzoquinone (purity 97%) and 6 ml toluene and 6 ml of the aqueous solution of the respective base in a concentration as indicated in table 3, in the presence of 3.5 mg of tetrabutylammonium bromide (1 mol % (relative to geranylgeranyl trimethyl benzoquinone)) were added and stirred under reflux (110° C.) for the time as indicated in table 3 to yield the 2,5,7,8-tetramethyl-2-(4,8,12-trimethyltrideca-3,7,11-trien-1-yl)-2H-chromen-6-ol (3,4-dehydro-α-tocotrienol) in conversion and yield as indicated in table 3.

TABLE 3
Aqueous solution of different bases as liquid phase base and toluene
forming a two-phase system liquid/liquid.

| Example | Base | Reaction time [h] | Conversion [%] | Yield [%] |
|---|---|---|---|---|
| 5 | NaOH (20% by weight in water) | 24 | 90 | 59 |
| 6 | NaOH (20% by weight in water) | 44 | 78 | 65 |
| 7 | KOH (20% by weight in water) | 20 | 75 | 54 |
| 8 | CsOH (20% by weight in water) | 42 | 83 | 67 |
| 9 | K₂CO₃ (20% by weight in water) | 44 | 84 | 78 |
| 10 | LiOH (10% by weight in water) | 40 | 54 | 47 |

### Partial Hydrogenation

3,4-Dehydro-α-tocotrienol (=2,5,7,8-tetramethyl-2-(4,8,12-trimethyltrideca-3,7,11-trien-1-yl)-2H-chromen-6-ol) as prepared above has been quantitatively hydrogenated to α-tocotrienol (=2,5,7,8-tetramethyl-2-(4,8,12-trimethyltrideca-3,7,11-trien-1-yl)chroman-6-ol according the procedure disclosed in the last paragraph on page 2524 of Schudel, Mayer, Isler, Helv. Chim. Acta 46, 2517-2526 (1963), the identity of which could be verified by NMR.

### Complete Hydrogenation

3,4-Dehydro-α-tocotrienol (=2,5,7,8-tetramethyl-2-(4,8,12-trimethyltrideca-3,7,11-trien-1-yl)-2H-chromen-6-ol) as prepared above has been quantitatively hydrogenated to α-tocophenol (=2,5,7,8-tetramethyl-2-(4,8,12-trimethyltridecyl)-chroman-6-ol) by hydrogen on Pd/C according to the last paragraph on page 888 of Kabbe and Heitzer, Synthesis 1978; 12, 888-889, the identity of which could be verified by NMR.

1. A process of manufacturing the compound of the formula (I) 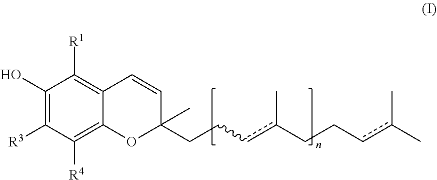
comprising a ring closing step of the compound of the formula (II) 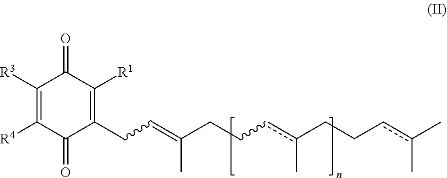
in the presence of a base in a two-phase system to yield the compound of the formula (I), wherein
n=0 or 1 or 2 or 3 or 4 or 5 or 6 or 7 or 8 or 9 or 10 or 11 or 12;
R¹ represents hydrogen or methyl groups;
R³ and R⁴
either represent independently from each other hydrogen or methyl group or methoxy group
or represent together a —CH—CH—CH— and form an aromatic group;
any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration.

2. The process according to claim 1, wherein R¹═R³═R⁴═CH₃.

3. The process according to claim 1 wherein the compound of the formula (I) is a compound of the formula (I-BB) and that the compound of the formula (ii) is a compound of the formula (II-BB)

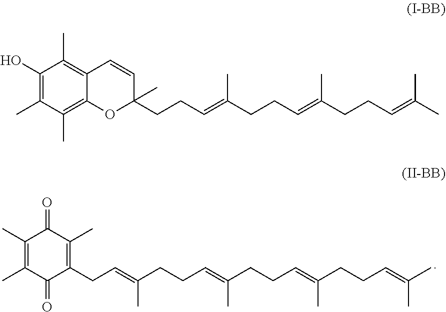

4. The process according to claim 1 wherein the base is in a different phase than the compound of the formula (II).

5. The process according to claim 1 wherein one of the two phases comprises a hydrocarbon, preferably toluene.

6. The process according to claim 1 wherein the base is present in an aqueous phase.

7. The process according to claim 1, wherein the base is in the form of a solid.

8. The process according to claim 1, wherein base is either an organic amine, preferably an organic tertiary amine, or a metal hydroxide or carbonate, particularly an organic tertiary amine or an alkali metal hydroxide.

9. The process according to claim 1 wherein the base is present in a molar ratio to the compound of the formula (I) of between 1:1′000 to 1:5, particularly 1:100 to 1:10.

10. The process according to claim 1 wherein the ring closing reaction is performed in the presence of a phase transfer agent, particularly of a quaternary ammonium salt, preferably of the formula [NR₄]X wherein R is a C₂₋₁₈-alkyl group, particularly a C₃₋₈-alkyl group, and X is a halide.

11. The process of manufacturing a compound of the formula (III) 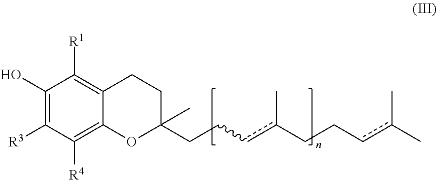
comprising the steps
a) manufacturing the compound of the formula (I) by a process according to claim 1 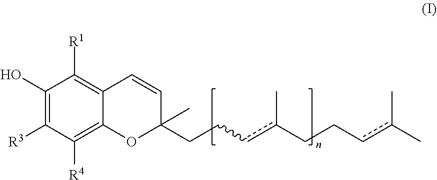
wherein any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration;
b) partially hydrogenating the compound of the formula (I) by means of a hydrogenating agent suitable for partial hydrogenation to yield the compound of the formula (III).

12. The process of manufacturing a compound of the formula (IV) 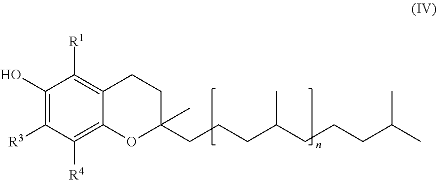
comprising the steps
a) manufacturing the compound of the formula (I) by a process according to claim 1 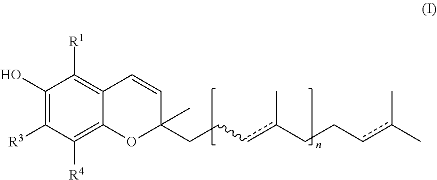
wherein any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration;
b′) hydrogenating the compound of the formula (I) by means of a hydrogenating agent to yield the compound of the formula (IV).

13. A two-phasic composition comprising
i)a compound of the formula (II) 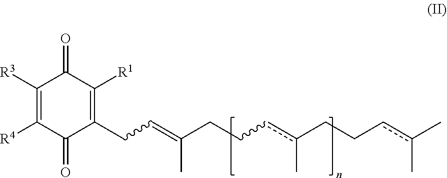
wherein
n=0 or 1 or 2 or 3 or 4 or 5 or 6 or 7 or 8 or 9 or 10 or 11 or 12;
R¹ represents hydrogen or methyl groups;
R³ and R⁴
either represent independently from each other hydrogen or methyl group or methoxy group
or represent together a —CH—CH—CH— and form an aromatic group;
any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration;
and
ii) a base in another phase than the compound of the formula (II).

14. The composition according to claim 13 wherein the compound of the formula (II) is a compound of the formula (II-BB)

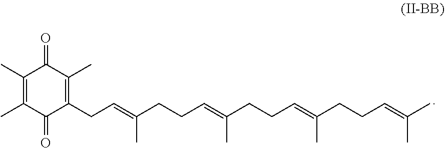 [SMILES: CC(C)=CCC/C(C)=C/CC/C(C)=C/CC/C(C)=C/CC1=C(C)C(=O)C(C)=C(C)C1=O]

15. Use of a base in a different phase than the compound of the formula (II) for the ring closure reaction of the compound of the formula (II) to yield the compound of the formula (I) 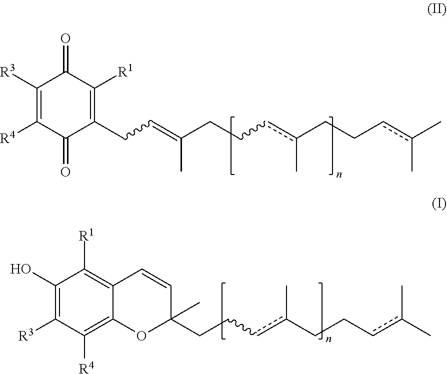
wherein
n=0 or 1 or 2 or 3 or 4 or 5 or 6 or 7 or 8 or 9 or 10 or 11 or 12;
R¹ represents hydrogen or methyl groups;
R³ and R⁴
either represent independently from each other hydrogen or methyl group or methoxy group
or represent together a —CH—CH—CH— and form an aromatic group;
any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration.

16. A use of the compound of the formula (I-A) or (I-C) or (III-A) or (III-C) or (IV-A) or (IV-C) as an antioxidant 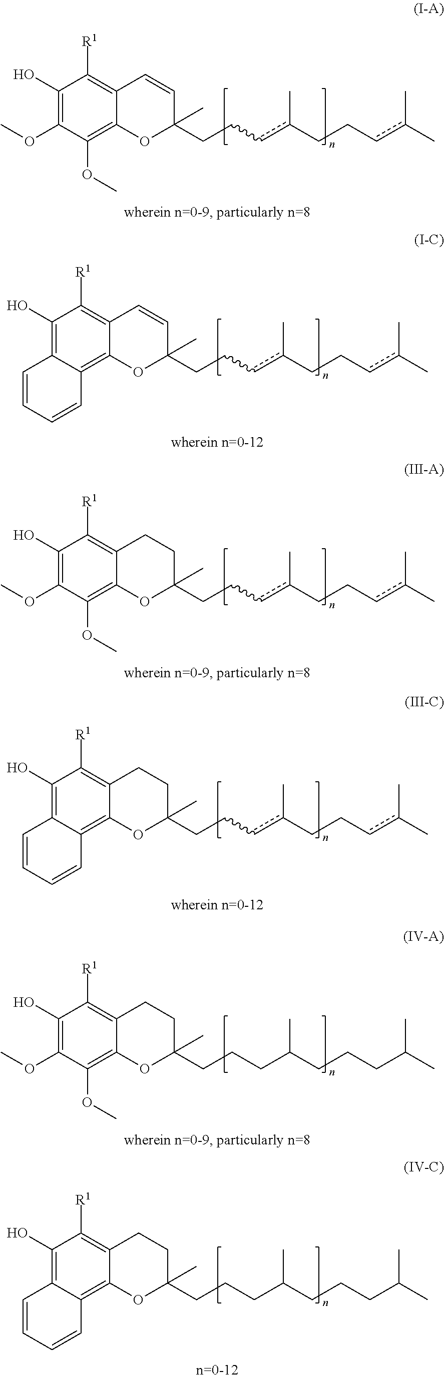
wherein
R¹ represents hydrogen or methyl groups;
any bond having dotted line ( [symbol] ) represents independently from each other either a carbon-carbon single bond or a carbon-carbon double bond; and
any wavy line represents independently from each other a carbon-carbon bond and which when linked to the carbon-carbon double bond is either in the Z or in the E-configuration.

17. A compound of the formula (I-As) or (I-Cis) or (III-Cis) or (IV-Cs) [chemical structure]
